(19) United States
(12) Patent Application Publication
(10) Pub. No.: US 2007/0149952 A1
(43) Pub. Date: Jun. 28, 2007

(54) Systems and methods for characterizing a patient's propensity for a neurological event and for communicating with a pharmacological agent dispenser

(76) Inventors: Mike Bland, Seattle, WA (US); Kent Leyde, Sammamish, WA (US); David Snyder, Bainbridge Island, WA (US); Daniel J. Dilorenzo, Seattle, WA (US)

Correspondence Address: SHAY LAW GROUP, LLP, 2755 CAMPUS DRIVE, SUITE 210, SAN MATEO, CA, 94403, US

(21) Appl. No.: 11/322,150

(22) Filed: Dec. 28, 2005

(51) Int. Cl. A61K 9/22

(52) U.S. Cl. 604/890.1

(57) ABSTRACT

The present invention provides systems and methods for managing intake of a pharmacological agent. In one method of the present invention, the systems and methods are for controlling intake of an anti-epileptic drug. In such embodiments, one or more signals from a patient are processed to predict an onset of a seizure. Upon the prediction of the seizure, the patient is allowed to access the pharmacological agent in a pharmacological agent dispenser.

### CROSS-REFERENCE TO RELATED APPLICATIONS

The present invention claims benefit to U.S. patent application Ser. No. ______, filed Dec. 28, 2005, entitled “Methods and Systems for Recommending an Appropriate Action to a Patient for Managing Epilepsy and Other Neurological Disorders,” (BNC Docket No. 2.00US; WSGR Docket No. 31685-713.201) and U.S. patent application Ser. No. ______, filed Dec. 28, 2005, entitled “Methods and Systems for Recommending a Pharmacological Treatment to a Patient for Managing Epilepsy and Other Neurological Disorders” (BNC Docket No. 2.01US; WSGR Docket No. 31685-713.202), both to Leyde et al., the complete disclosures of which are incorporated herein by reference.

### BACKGROUND OF THE INVENTION

The present invention relates generally to monitoring a patient's condition and controlling administration of a pharmacological agent. More specifically, the present invention is directed to characterizing a patient's propensity for a future seizure and facilitating access to and administration of an anti-epileptic drug.

Epilepsy is a disorder of the brain characterized by chronic, recurring seizures. Seizures are a result of uncontrolled discharges of electrical activity in the brain. A seizure typically manifests as sudden, involuntary, disruptive, and often destructive sensory, motor, and cognitive phenomena. Seizures are frequently associated with physical harm to the body (e.g., tongue biting, limb breakage, and burns), a complete loss of consciousness, and incontinence. A typical seizure, for example, might begin as spontaneous shaking of an arm or leg and progress over seconds or minutes to rhythmic movement of the entire body, loss of consciousness, and voiding of urine or stool.

A single seizure most often does not cause significant morbidity or mortality, but severe or recurring seizures (epilepsy) results in major medical, social, and economic consequences. Epilepsy is most often diagnosed in children and young adults, making the long-term medical and societal burden severe for this population of patients. People with uncontrolled epilepsy are often significantly limited in their ability to work in many industries and cannot legally drive an automobile. An uncommon, but potentially lethal form of seizure is called status epilepticus, in which a seizure continues for more than 30 minutes. This continuous seizure activity may lead to permanent brain damage, and can be lethal if untreated.

While the exact cause of epilepsy is uncertain, epilepsy can result from head trauma (such as from a car accident or a fall), infection (such as meningitis), or from neoplastic, vascular or developmental abnormalities of the brain. Most epilepsy, especially most forms that are resistant to treatment (i.e., refractory), is idiopathic or of unknown causes, and is generally presumed to be an inherited genetic disorder. Demographic studies have estimated the prevalence of epilepsy at approximately 1% of the population, or roughly 2.5 million individuals in the United States alone. Approximately 60% of these patients have epilepsy where a defined point of onset can be identified in the brain and are therefore candidates for some form of a focal treatment approach.

While there is no known cure for epilepsy, usage of anticonvulsant and antiepileptic medications has been relatively effective in controlling seizures in most people. The anticonvulsant and antiepileptic medications do not actually correct the underlying conditions that cause seizures. Instead, the anticonvulsant and antiepileptic medications (referred to herein collectively as an “AED”) manage the patient's epilepsy by reducing the frequency of seizures. There are a variety of classes of AEDs, each acting by a distinct mechanism or set of mechanisms. For most cases of epilepsy, the disease is chronic and requires chronic medications for treatment. AEDs generally suppress neural activity by a variety of mechanisms, including altering the activity of cell membrane ion channels and the propensity for action potentials or bursts of action potentials to be generated. Some of the fast acting AEDs are primarily used as sedatives, and their desired therapeutic effects are often accompanied by the undesired side effect of sedation. Other medications have significant non-neurological side effects, such as gingival hyperplasia, a cosmetically undesirable overgrowth of the gums, and/or a thickening of the skull, as occurs with Phenyloin. While chronic usage of AEDs has proven to be relatively effective for a majority of patients suffering from epilepsy, the persistent side effects can cause a significant impairment to a patient's quality of life. Furthermore, about 30% of epileptic patients are refractory (e.g., non-responsive) to the conventional chronic AED regimens. This creates a scenario in which over 500,000 patients in the United States alone have uncontrolled epilepsy.

Because of the severe side effects caused by the chronic administration of high dosages of AEDs, patient compliance with the chronic AED regimen has proven to be a difficult problem to overcome. Consequently, many patients are still prone to seizures due to the noncompliance with their chronic AED regimen.

Equally problematic is the fact that many AEDs are highly addictive, and clinicians are hesitant to prescribe certain AEDs for fear of chemical abuse of such AEDs. Thus, many patients are not being prescribed what could be the most effective therapy, due to fears of drug abuse.

Consequently, what are needed are methods and systems which provide for improved AED treatments for patients to manage their epilepsy. It would be desirable to be reduce the potential for abuse of the AEDs, while providing reduced side effects caused by the AEDs.

### SUMMARY OF THE INVENTION

The present invention addresses the problem of chemical abuse of AEDs by providing a way to selectively limit the access to and administration of the AEDs. The systems and methods of the present invention are able to characterize the patient's propensity or likelihood of a future seizure. Upon determination of an increased propensity or likelihood of the future seizure, the present invention facilitates allows the patient to access the AED. In preferred embodiments, the patient is only allowed to access a dosage of the AED that is needed to prevent the seizure. The ability of the present invention to deliver an AED at substantially the lowest effective dose and at or near the time when the patient has an elevated propensity would minimize the exposure of the patient to side effects, maximize the benefit of the AED and help limit the potential chemical abuse of the AED.

As used herein, the term “anti-epileptic drug” or “AED” generally encompasses pharmacological agents that have been determined to reduce the frequency or propensity for a seizure. There are many drug classes that comprise the set of antiepileptic drugs (AEDs) that may be used by the present invention, and many different mechanisms of action are represented. For example, some medications are believed to increase the seizure threshold, thereby making the brain less likely to initiate a seizure. Other medications retard the spread of neural bursting activity and tend to prevent the propagation or spread of seizure activity. Some AEDs, such as the Benzodiazepines, act via the GABA receptor and globally suppress neural activity. However, other AEDs may act by modulating a neuronal calcium channel, a neuronal potassium channel, a neuronal NMDA channel, a neuronal AMPA channel, a neuronal metabotropic type channel, a neuronal sodium channel, and/or a neuronal kainite channel, and all are encompassed by the present invention.

One or more parameters of the AED that is administered to the patient are preferably titrated to correspond to the patient's propensity or likelihood for the future seizure. Titration of the AED is typically predetermined by a clinician so that the administered AED is commensurate with the patient's propensity or likelihood of the seizure. For example, if the patient's propensity for a future seizure is low (e.g., a long time horizon is predicted), the initial amount of AED that is administered will typically be lower than a “normal” dosage, the formulation will not necessarily be fast acting, and/or the AED will have a lower side effect profile. But if the patient's propensity for a future seizure is high (e.g., a short time horizon is predicted, high probability, etc.), the dosage of AED that is administered will likely be higher than the normal dosage and/or the form, formulation, and route of administration may be selected to be fast acting. Depending on the propensity or likelihood, it may be desirable to administer a different form, formulation, or provide a route of administration for the AED.

While titration of the AED to be a function of the patient's propensity for a seizure is a preferred embodiment, in alternative embodiments, the present invention may simply provide a single standard dosage for the patient. While the dosages may not be optimized to be a function of the patient's propensity for a seizure, such dosages would still likely be administered acutely and would not require the patient to chronically administer the AED. The acute dosage could be a reduced dosage, a normal dosage, or an increased dosage relative to the dosage of the chronically administered AED.

In other alternative embodiments, the patient may be maintained on a chronic regimen of AEDs, in which the dosages may be at conventional dosages or sub-conventional dosages, and the present invention may be used to augment the AED regimen by monitoring the patient's propensity for a seizure and providing additional acute, preventative dosages of AEDs when it is determined that the patient's propensity for a seizure is elevated. The acute dosage could be a reduced dosage, a normal dosage, or an increased dosage relative to the dosage of the chronically administered AED.

Such a paradigm has many advantages over conventional AED treatments, including the ability to administer an AED at or near the optimal dose and only when the AED is actually needed (e.g., when the patient's propensity for seizure is elevated). Consequently, the present invention minimizes the patient's exposure to the undesirable side effects of the AED. Moreover, even if the dosage is the same as the chronic dosages or the seizure breaking dosage, the side effects caused by the present invention would be limited in duration (instead of chronically having the side effects).

The AEDs may be housed in a pharmacological agent dispenser that is implanted in the patient's body, external to the patient's body, or any combination thereof. The dispenser may be in any form known to those of ordinary skill in the art, but is typically in the form of a pill dispenser, a metered dose inhaler, an external or internal drug pump, an intravenous (IV) drug delivery assembly, intramuscular drug delivery assembly, a transcutaneous or subcutaneous drug delivery system, an implanted access port, intraventricular drug delivery systems, intrathecal drug delivery systems, intraparenchymal drug delivery systems, or the like.

In preferred embodiments, the drug dispenser is in one-way or two-way communication with at least one component of the system of the present invention. The drug dispenser may be in communication with a device that is external to the patient's body and/or a device that is implanted in the patient's body. The communication link between the dispenser and other components of the system may be used to control the administration of the AED to the patient. In preferred embodiments, when it is determined that the patient is at an elevated propensity for a seizure, the communication link is used to allow the patient to access to the dispenser or enable a patient or caregiver to dispense the drug (e.g., unlock the dispenser). The communication link may also be used to prevent access to the dispenser (e.g., lock the dispenser), control the rate of access, control the amount time that the drug is accessible by the patient, control the amount of drug that is administered, and the like.

Controlling access to the AED has a number of advantages. First, it provides a way of reducing, and preferably preventing, abuse of the AEDs by allowing the patient to access the AED only when it is determined that the patient has an elevated propensity for a seizure. Second, it allows administration of only the amount of AED needed to prevent or otherwise manage the predicted future seizure. Such titration may be done automatically by the system or the patient may be instructed to titrate.

Communication with the dispenser may also allow other components of the system to monitor the amount of drugs that has been accessed or administered by the patient. Monitoring may be carried out by measuring the number of times the pharmacological agent was made accessible over a time period, measuring the number of times the pharmacological agent dispenser was activated, measuring the number of times the patient affirmed that the pharmacological agent was taken, monitoring the patient's propensity for seizure for an expected response to the pharmacological agent (e.g., change from an elevated propensity for a seizure state back to a normal state), or a combination thereof. By knowing the amount of drug that has been administered, the clinician and/or the system may be able to track the patient's intake, compliance with the clinician's recommendations, effectiveness of the drug in perturbing the propensity for seizures, etc. Advantageously, such data may be used by the clinician and or system to adapt future seizure likelihood determinations and adapt the dosages or selection of AEDs that are administered to the patient. Furthermore, if it is determined that a maximum threshold of the selected AED (or AEDs) has been reached, the systems and methods of the present invention may be configured prevent access to the AED and recommend or provide an alternate treatment for preventing the therapy. The alternate therapy may include a different AED, a non-pharmacological agent, electrical stimulation, or other action.

While the AED may be automatically administered to the patient with an implanted or external drug pump, in preferred embodiments, administration of the pharmacological agent is carried out through manual actuation of the unlocked pharmacological agent dispenser. Administration of the AED to the patient may be facilitated by the systems of the present by providing a communication to the patient. Typically, the communication is provided through a handheld patient communication assembly. The patient communication assembly may be physically attached to or a part of the drug dispenser or the patient communication assembly may altogether be a separate device from the dispenser. The patient communication assembly may comprise one or more output assemblies that may provide a visual output (such as text, lights, or other images), an audio output (such as one or more beeps or voice instructions), a mechanical output (such as a vibration), or the like.

The communication output to the patient may include any type of information that is desired by the clinician or patient, however, the communication is typically indicative of the patient's propensity for a future seizure and will be provided to the patient at an appropriate time, such as when it is determined that the patient has an increased propensity for a seizure. Of course, if desired, outputs may be provided substantially continuously to the patient to provide an indication of their state or propensity for seizure, so that the patient will have a real-time understanding of their state. When there is a fluctuation in a neural state that indicates an increased propensity for a seizure, a warning may be provided to the patient to indicate the state change.

In some embodiments, the communication to the patient provides a recommendation to the patient regarding the appropriate action for preventing or managing the predicted seizure. The recommendation is typically predetermined by a clinician and the recommendation will be a function of the patient's measured propensity for the seizure. The communication typically will recommend that the patient take a pharmacological agent. In some embodiments, the recommendation will be titrated to the patient's increased propensity for the future seizure. As such, depending on the patient's propensity, the recommendation may indicate at least one of a dosage, form of the drug, formulation of the drug, and route of administration. In other embodiments, however, the recommendation may just instruct the patient to “take your AED” or otherwise warn the patient, and the patient will know to take a single “normal” dosage of the patient's prescribed drug(s).

The communication to the patient that is indicative of an appropriate action is not limited to recommending or instructing the patient to take a pharmacological agent. An instruction to perform any accepted means for managing or treating epileptic seizures may be output to the patient. For example, if the seizure is imminent and is likely not to be averted with electrical stimulation or pharmacological agents, the communication may simply warn the patient of the imminent seizure and simply instruct the patient to “make yourself safe.” This would allow the patient to stop driving, lie down, stop cooking, or the like. Some additional recommendations or instructions that may be provided to the patient include, but are not limited to, turning off lights, interrupting work, touching the face, hyperventilating, hypoventilating, holding breath, performing the valsalva maneuver, applying an external stimulator (e.g., lights, electrical stimulation, etc.), applying transcutaneous electrical neurostimulation, applying tactile stimulation, activating an implanted deep brain neurostimulator, activating an implanted vagus nerve stimulator, activating another neuromodulator, activating an implanted drug pump, begin taking one or more medications, stop taking medications, increase or reduce medication dosage, change medication dosing regimen, and other initiation of action, change of behavior, or cessation of activity.

Characterization of the patient' propensity for a future seizure may be carried out in any number of ways. In one embodiment, the patient's propensity for a future seizure is derived at least in part from a patient's neural state, which can be characterized as a patient's state along a single or multi-variable state space continuum. The term “neural state” is used herein to generally refer to calculation results or indices that are reflective of the state of the patient's neural system, but does not necessarily constitute a complete or comprehensive accounting of the patient's total neurological condition. The estimation and characterization of “neural state” may be based on one or more patient signals from the brain, including but not limited to electroencephalogram signals “EEG” and electrocorticogram signals “ECoG” (referred to herein collectively as “EEG”), brain temperature, blood flow in the brain, concentration of AEDs in the brain, etc.).

In addition to using the neural state, the propensity for seizure may also be derived using other patient dependent parameters, such as patient history, and/or other physiological signals from the patient. Some of the physiological signals that may be monitored include, temperature signals from other portions of the body, blood flow measurements in other parts of the body, heart rate signals and/or change in heart rate signals, respiratory rate signals and/or change in respiratory rate signals, chemical concentrations of other medications, pH in the blood or other portions of the body, blood pressure, other vital signs, other physiological or biochemical parameters of the patient's body, or the like).

The methods and systems of the present invention may also have the capability to use feedback from the patient as an additional metric for characterizing the patient's propensity for a seizure. For example, in some embodiments, the system may allow the patient to affirm that the AED was taken, indicate that they didn't take the AED, indicate that they are feeling an aura or are experiencing a prodrome or other symptoms that precede a seizure, indicate that they had a seizure, indicate that they are going to sleep or waking up, engaging in an activity that is known to the patient to interfere with their state, or the like.

A neural state index, which may be displayed to the patient or caregiver, may be a derivative of the neural state and the other patient dependent parameters or a simplified output of measurements performed by a predictive algorithm. The neural state index may be simplified to one or more scalar numbers, one or more vectors, a symbol, a color, or any other output that is able to differentiate variations of the patient's neural state.

In one embodiment, the present invention processes the one or more signals using a predictive algorithm. The predictive algorithm typically comprises one or more feature extractors and a classifier. The feature extractors typically extract one or more features from the patient dependent parameters. The features typically include mathematical features derived from the brain signals and other physiological features. At least one of the extracted features, and preferably a plurality of the extracted features are sent to a classifier to characterize the patient's propensity for a future seizure.

The classifier is configured to combine the results obtained from the feature extractors and other signals into an overall answer or result, which classifies the patient's state and characterizes the patient's propensity for the future seizure. The classifier may provide a simple characterization that the patient is at an increased risk of a seizure, e.g., the patient's neural state has changed from a normal (e.g., inter-ictal state) to a state that is consistent with a predetermined state such as a “an elevated propensity for seizure state” or “pre-ictal” state (e.g., a state that precedes an “ictal” or seizure state). Alternatively, the classifier may provide a graded answer that that would allow for estimation of a prediction interval (e.g., 30 seconds or more, 1 minute or more, 2 minute or more, 5 minutes or more 10 minutes or more, 30 minutes or more, 60 minutes or more, or the like), characterization of a graded response that is a function of the graded answer, or the like.

The feature extractors and classifier modules of the predictive algorithm may be embodied in a device that is implanted in a patient, embodied in a device that is external to the patient, or in a combination thereof. For example, in one configuration, both the feature extractors and classifier are embodied within a device assembly that is implanted in the patient. In another configuration, both the feature extractors and classifier may be embodied in a device that is external to the patient's body, such as in a patient communication assembly. In yet other configurations, one of the feature extractor and classifier is embodied in a device assembly that is implanted in the patient's body, while the other of the classifier and feature extractor is embodied in a device that is external to the patient's body.

While the remaining discussion focuses characterizing a patient's neural state to predict an onset of future seizures and providing a communication link with an AED dispenser, it should be appreciated that the present invention may be used to monitor other neurological and non-neurological conditions and facilitate the administration of other therapies besides pharmacological agents. For example, the present invention may monitor the cardiac system and be used to titrate a patient's heart medication, monitor glucose levels and control administration of insulin, or monitor other neurological conditions (e.g., depression, Parkinson's disease, or the like) and provide for controlled administration of the related drugs. Advantageously, by predicting the occurrence of some event and facilitating controlled administration of a pharmacological agent, the present invention is able to control the disorder, while reducing the side effects caused by the pharmaceutical agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A schematically illustrates a simplified system of the present invention that includes a patient interface assembly, a device assembly, a patient communication assembly and a pharmacological agent dispenser.

FIG. 1B schematically illustrates a simplified system of the present invention that includes a patient interface assembly, a processing device, and an integrated patient communication assembly and pharmacological agent dispenser.

FIG. 2 illustrates one preferred system that is encompassed by the present invention.

FIG. 3 illustrates a simplified device assembly that is encompassed by the present invention.

FIG. 4 illustrates a predictive algorithm that is encompassed by the present invention.

FIGS. 5A to 5E illustrate various embodiments of a predictive algorithm and treatment algorithm.

FIG. 6 provides a block diagram of one exemplary pharmacological agent dispenser that is encompassed by the present invention.

FIG. 7 is a flow chart that illustrates a method that is encompassed by the present invention.

FIG. 8 illustrates a kit that is encompassed by the present invention.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

### DETAILED DESCRIPTION OF THE INVENTION

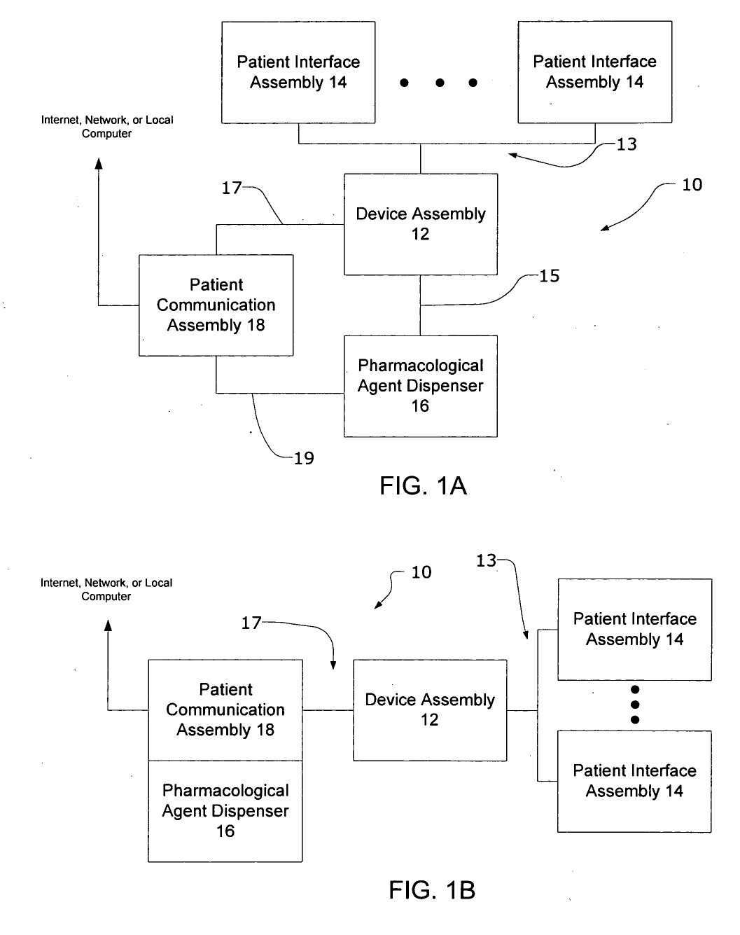

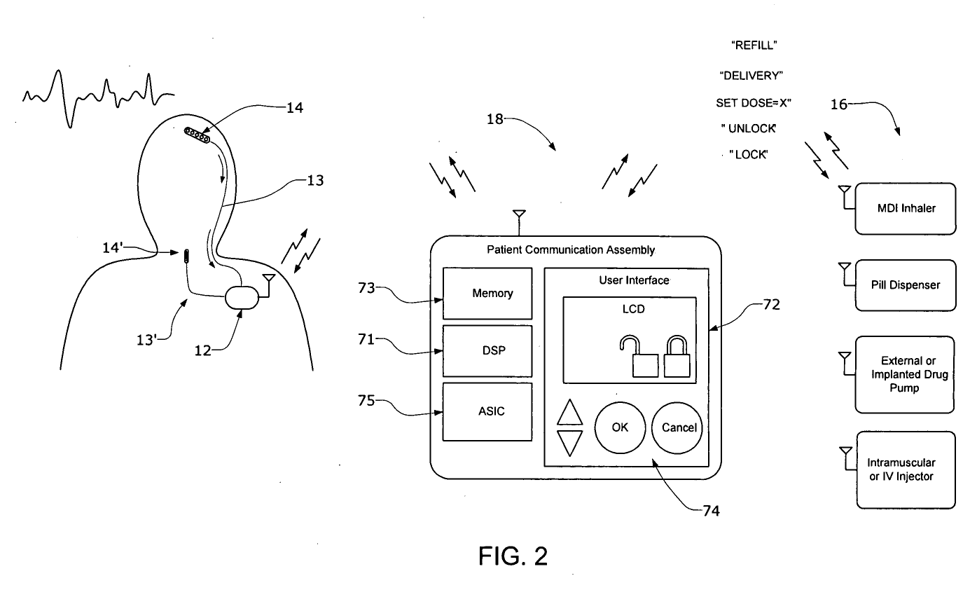

FIG. 1A illustrates a simplified system 10 that is encompassed by the present invention. System 10 includes a device assembly 12 that is coupled to one or more patient interface assemblies 14 with a communication link 13. For ease of reference, device assembly 12 is illustrated as a single component, but it should be appreciated that device assembly 12 may be comprised of multiple components that may be implanted within a patient's body, external to the patient's body, or a first component of the device assembly may be implanted and a second component of device assembly may be external to the patient's body.

Patient interface assembly 14 may be configured to sense one or more signals from the patient, deliver therapy to the patient, or both. Patient interface assembly 14 illustrated to in FIG. 1A typically includes a plurality of electrodes, thermistors, physiological sensors, or other sensors as known in the art. In preferred embodiments, the patient interface assembly 14 comprises a plurality of electrodes for sensing physiological parameters from the patient. While the patient interface assembly 14 may include any number of electrodes, it typically has between about 1 electrode and about 64 electrodes, and preferably between about 2 electrodes and about 8 electrodes. The electrodes may be in communication with a nervous system component (which is used herein to refer to any component or structure that is part of or in communication with the nervous system), a non-nervous system component, or a combination thereof.

Patient interface assembly 14 typically includes an array of intracranial EEG electrodes that are either in a subgaleal location within or below the scalp and above the skull or beneath the skull, each of which facilitates communication with some portion of the patient's nervous system. It should be appreciated however, that the intracranial electrode may be placed in other portions of the patient's head. Some additional useful areas for placing the intracranial electrodes include, but are not limited to, the hippocampus, amygdala, anterior nucleus of the thalamus, centromedian nucleus of the thalamus, other portion of the thalamus, subthalamic nucleus, motor cortex, premotor cortex, supplementary motor cortex, other motor cortical areas, somatosensory cortex, other sensory cortical areas, Wernicke's Area, Broca's Area, pallido-thalamic axons, lenticulo-thalamic fiber pathway, substantia nigra pars reticulata, basal ganglia, external segment of globus pallidus, subthalalmic to pallidal fiber tracts, putamen, putamen to PGe fibers, other areas of seizure focus, other cortical regions, or combinations thereof.

In addition to being placed intracranially, the patient interface assembly 14 may be placed extracranially and in communication with an extracranial nervous system component, such as a peripheral nerve or cranial nerve, (e.g., the vagus nerve, olfactory nerve, optic nerve, oculomotor nerve, trochlear nerve, trigeminal nerve, abducens nerve, facial nerve, vestibulocochlear nerve, glossopharyngeal nerve, accessory nerve, hypoglossal nerve) or it may be coupled to other portions of the patient's body, such as to an external surface of the patient's cranium (e.g., above, below, or within the patient's scalp).

In addition to or as an alternative to the EEG electrode array, patient interface assembly 14 may comprise electrodes or other sensors that are configured to sense other physiological signals from the patient. Some examples of such signals include but are not limited to, electromyography (EMG) signals, electrocardiogram (ECG) signals, temperature signals from the brain or other portions of the body, blood flow measurements in the brain and/or other parts of the body, heart rate signals and/or change in heart rate signals, respiratory rate signals and/or change in respiratory rate signals, chemical concentrations of AED or other medications, pH in the brain, blood, or other portions of the body, blood pressure, or other vital signs or physiological parameters of the patient's body.

As noted above, one or more of the patient interface assembly 14 may also be used to deliver a therapy to the patient. The therapy may comprise an electrical, thermal, or optical therapy that is delivered to a nervous system component or non-nervous system component of the patient. In such embodiments, patient interface assembly 14 may comprise one or more stimulation electrodes. As is known in the art, such patient interface assembly 14 may be implanted within the patient's body or positioned external to the patient's body.

System 10 further comprises a pharmacological agent dispenser 16 that is in communication with device assembly 12 via a communication link 15. The dispenser 16 may be used to systemically deliver a pharmacological agent to the patient or it may be used to locally deliver the pharmacological agent (e.g., directly to the seizure focus or other appropriate site). Signals from device assembly 12 may facilitate initiation of a pharmacological therapy via the pharmacological agent dispenser 16. In preferred embodiments, pharmacological agent dispenser 16 is external to the body of the patient and is manually activatable. But in other embodiments, the pharmacological agent dispenser may be implanted in the body of the patient and/or automatically controlled by device assembly 12.

For example, an implanted pharmacological agent dispenser 16 may be used to directly infuse therapeutic dosages of one or more pharmacological agents into the patient, and preferably directly into affected or associated portion(s) of the brain. The medications will generally either decrease/increase excitation or increase/decrease inhibition. Consequently, the type of drugs infused and the patient's disorder will affect the area in which the medication dispenser is placed. Some examples of medication dispensers that can be used with the system of the present invention are described in U.S. Pat. Nos. 6,094,598, 5,735,814, 5,716,377, 5,711,316, and 5,683,422. In some embodiments of the present invention, the dosage and/or timing of the medication delivery may be varied depending on the processing performed by device assembly 12. Implanted medication reservoirs may be used, including intracranial, intraventricular (in the cerebral ventricle), intrathecal, intravenous, and other catheters. Such embodiments include indwelling central venous catheters for rapid administration as well as peripheral venous catheters.

System 10 may optionally include a patient communication assembly 18 that is in communication with at least one of the device assembly 12 and pharmacological agent dispenser 16 via communication links 17, 19, respectively. Patient communication assembly 18 may be used as a user interface to provide a one-way or two way communication between the system and the patient, and act as an indirect communication link between a device assembly 12 and pharmacological agent dispenser 16. In some embodiments, patient communication assembly 18 may be used to process data signals from patient interface assembly 14 and when needed, enable access to pharmacological agent dispenser 16. In other embodiments, however, patient communication assembly 18 may provide minimal processing and be used primarily to facilitate communication between the system and the patient.

Communication links 13, 15, 17, 19 illustrated in FIG. 1A may be any combination of wired or wireless communication links and include conventional communication protocols known to those of ordinary skill in the art, including but not limited to telemetry, inductive coil links, RF links, other electromagnetic links, magnetic links, infrared links, optical links, ultrasound links, or the like.

While not illustrated in FIG. 1A, system 10 may also optionally include a clinician communication assembly that may be put into direct or indirect communication with device assembly 12. For example, clinician communication assembly may communicate with device assembly 12 with a direct communication link, or may communicate with device assembly 12 indirectly through patient communication assembly 18 (or another communication device (not shown)). Clinician communication assembly may also be in communication with a personal computer to allow for download or upload of information from clinician communication assembly, or configuration or reprogramming of the clinician communication assembly, patient communication assembly 18, device assembly 12, or the like. Clinician communication assembly and the personal computer may allow a patient's guardian or clinician to remotely monitor the patient's neural state, propensity for seizure, and/or medication intake in a real-time or non-real time basis.

System 10 may also have the capability to directly or indirectly connect to the Internet, a wide area network, a local area network, or a local computer, so as to allow uploading or downloading of data, programming parameters, or the like, to and from patient communication assembly 18 or clinician communication assembly to a remote server or database, or to allow a clinician or supervisor to remotely monitor the patient's neural state and/or propensity for seizure on a real-time or non-real-time basis. Connection to the Internet may be carried out through connection to a personal computer, but in other embodiments, it may be possible to directly connect to the Internet through a communication port on patient communication assembly 18, clinician communication assembly, dispenser 16, or device assembly 12. A more complete description of other features of systems that may be used to measure a patient's propensity for a seizure and provide communications to the patient may be found in U.S. patent application Ser. No. ______, filed Dec. 28, 2005, entitled “Methods and Systems for Recommending an Appropriate Action to a Patient for Managing Epilepsy and Other Neurological Disorders,” (BNC Docket No. 2.00US; WSGR Docket No. 31685-713.201) and U.S. patent application Ser. No. ______, filed Dec. 28, 2005, entitled “Methods and Systems for Recommending a Pharmacological Treatment to a Patient for Managing Epilepsy and Other Neurological Disorders” (BNC Docket No. 2.01US; WSGR Docket No. 31685-713.202), both to Leyde et al., U.S. Pat. Nos. 6,366,813 and 6,819,956 and U.S. patent application Ser. No. 10/753,205 (filed Jan. 6, 2004), Ser. No. 10/818,833 (filed Apr. 5, 2004), Ser. No. 10/858,899 (filed Jun. 1, 2004), Ser. No. 10/889,844 (filed Jul. 12, 2004), and Ser. No. 11/159,842 (filed Jun. 22, 2005).

The systems 10 of the present invention may be modified to combine components with each other. For example, FIG. 1B illustrates an embodiment in which the patient communication assembly 18 and the pharmacological agent dispenser 16 are a single assembly that has a wired communication link between them. In such embodiments, the device assembly will typically be implanted in the patient and the patient will be responsible for maintaining only a single device. Moreover, because the two components 16, 18 are integrated with each other, a single communication link 17 may be provided between the combined assembly and the implanted device assembly 12. Furthermore, instead of having separate processors/memories for each component, a single processor and memory may be used to control the combined assembly. The combined assembly will typically have the same functionality as the other embodiments described herein. While not shown, in other alternative embodiments it may be possible to combine the device assembly 12 and patient communication assembly 18 into a single assembly. While the remaining discussion focuses primarily on wireless communication between the various separate components of the system, it should be appreciated that the components of the system may be integrated together and the communications may be carried out through wired communications.

In use, if patient interface assembly 14 is used for sensing signals from the patient, signal(s) from patient interface assembly 14 are typically transmitted over communication link 13 to device assembly 12 where the measured signal(s) are processed in order to characterize a patient's propensity for a future seizure. As will be described below, processing of the signals typically comprises characterizing a patient's neural state which may be used to at least partially characterize the patient's propensity for a seizure. The characterization of the patient's propensity for seizure may be used in a number of different ways. For example, the present invention may provide a communication output to at least one of the patient communication assembly 18 and the pharmacological agent dispenser 16 over communication links 15, 17, 19. The communication output may provide any combination of a warning to the patient, instruction or recommendation to the patient, providing data to the patient regarding their neural state or propensity for a future seizure, enabling usage or access to the pharmacological agent in the dispenser 16, controlling the dispenser, or the like.

The communication output provided to the patient will typically be a function of the patient's propensity for seizure, and will typically indicate an appropriate treatment for preventing the future seizure. Likewise, the communication output to the dispenser 16 may allow the patient to access a pharmacologically effective amount of agent to prevent the future seizure. In alternative embodiments, however, the communication to the patient communication assembly may simply be a warning and the communication output to the dispenser 16 may simply allow the patient to access the dispenser (with or without titrating the dosage of the agent).

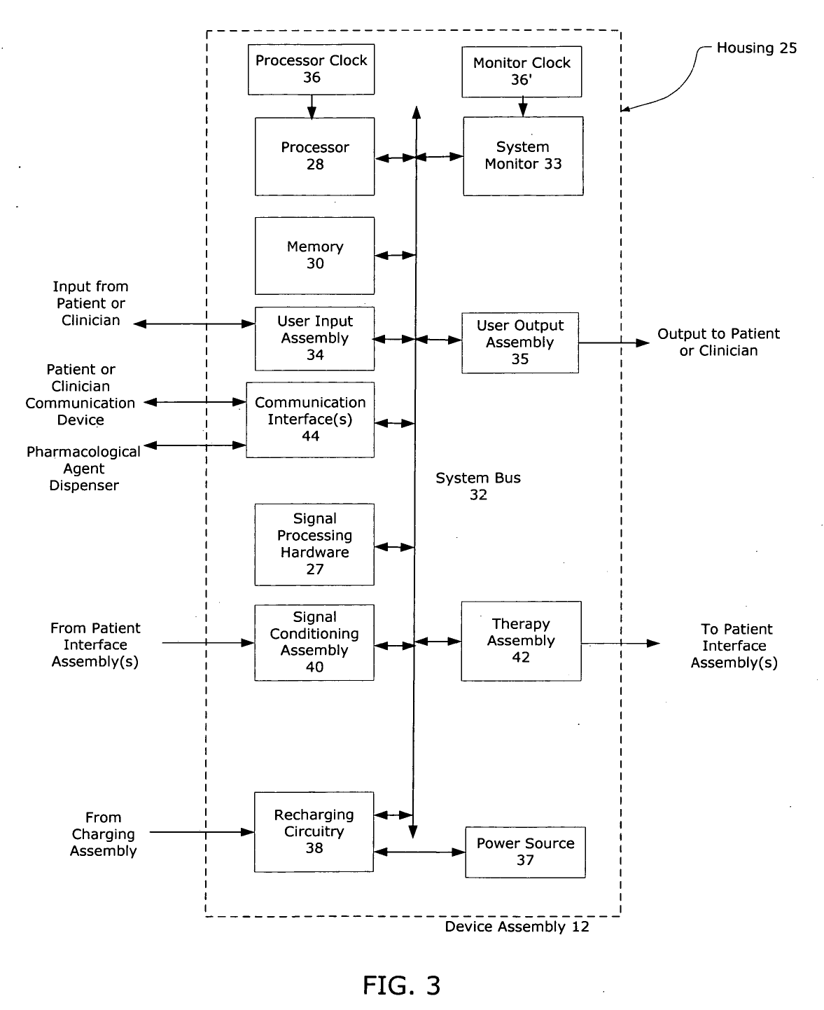

FIG. 2 illustrates one preferred system 10 encompassed by the present invention. In the illustrated embodiment of FIG. 2, patient interface assembly 14 is in the form of an intracranial EEG sensor array, and the device assembly 12 is implanted in a sub-clavicular cavity in the patient's body. Communication between the intracranial patient interface assembly 14 and device assembly 12 is carried out with a wired electrical communication link 13 that is tunneled from the device assembly 12 to the intracranial sensor array 14. While not shown, in other embodiments, it may be possible to provide a wireless link between device assembly 12 and intracranial sensor array 14. System 10 may optionally include an extracranial patient interface assembly 14′ coupled to device assembly 12 via a wired electrical communication link 13′. Patient interface assembly 14′ is typically in the form of stimulation electrodes that are configured to be coupled to a peripheral nerve, such as the vagus nerve.

System 10 includes patient communication assembly 18 that is external to the patient's body and is in wireless communication with device assembly 12. Patient communication assembly 18 may be used to process signals received from patient interface assembly 14 to characterize the patient's propensity for a future seizure, deliver the communication output to the patient and/or to allow the patient to provide inputs into system 10.

As shown in FIG. 2, patient communication assembly 18 is typically a handheld device that comprises a housing for storing the components of assembly 18. Patient communication assembly typically comprises a processor 71 (such as a digital signal microprocessor) that is in communication with a memory 73 and an application specific integrated circuit (ASIC) 75. The ASIC may be a custom integrated circuit that is programmed to calculate select portions of the prediction algorithm, treatment algorithm or other functions of the patient interface assembly. Memory 73 may be in the form of a Flash card or a small hard drive and may be used to store selected aspects of the patient's status, monitor the detected events, patient input events, or the like. Such data may be stored in memory and automatically transmitted to a host computer, network, over the internet or the like. Alternatively, such data may be stored until the clinician uploads such data from memory. A more detailed description of some useful patient communication assemblies 18 may be found in U.S. patent application Ser. No. ______, filed Dec. 28, 2005, entitled “Methods and Systems for Recommending an Appropriate Action to a Patient for Managing Epilepsy and Other Neurological Disorders,” (BNC Docket No. 2.00US; WSGR Docket No. 31685-713.201) and U.S. patent application Ser. No. ______, filed Dec. 28, 2005, entitled “Methods and Systems for Recommending a Pharmacological Treatment to a Patient for Managing Epilepsy and Other Neurological Disorders” (BNC Docket No. 2.01US; WSGR Docket No. 31685-713.202), both to Leyde et al.

As shown in FIG. 2, the patient communication assembly 18 typically comprises a user interface that comprises outputs 72 such as auditory devices (e.g., speakers) visual devices (e.g., LCD display), tactile devices (e.g., vibratory mechanisms), or the like, and inputs 74, such as a plurality of buttons, a touch screen, and a scroll wheel. As shown in FIG. 2, the LCD may be used to output a variety of different communications to the patient including, but not limited to, the state of the dispenser (e.g., locked or unlocked), battery state of one or more components of system 10, status of the pharmacological agent supply in dispenser 16, a warning (e.g., “Seizure warning”), a recommendation (e.g., “Take drugs”), a recommended dosage (e.g., “take 20 μg of drug”), or the like. Of course, it may be desirable to provide an audio output or vibratory output to the patient in addition to or as an alternative to the visual display on the LCD.

The manually activatable pharmacological agent dispenser 16 comprises a communication port that facilitates wireless communication with patient communication assembly 18, device assembly 12, or both. The pharmacological agent dispenser 16 of the present invention includes a variety of different types of dispensers. For example, the dispenser may include, but is not limited to, a pill dispenser, a metered dose inhaler, an external or internal drug pump, an intravenous (IV) drug delivery assembly, an intramuscular injector, a transcutaneous or subcutaneous drug delivery system, or the like. The communication signal received from patient communication assembly 18 and/or device assembly 12 allows the patient to access the pharmacological agent within dispenser 16. In some embodiments, the communication signal may adapt dispenser 16 to allow administration only of the appropriate dosage. In other embodiments, the communication signal may simply enable the patient to administer the pharmacological agent (e.g., unlock the dispenser 16).

In addition to or as an alternative to a communication output to the patient and dispenser 16, upon determining that that patient has an increased propensity for a future seizure, device assembly 12 may optionally generate a therapeutic output signal that is reflective of an appropriate therapy for mitigating the patient's increased propensity for the future seizure. The appropriate treatment may be automatically delivered to the appropriate patient interface assembly 14′, such as a stimulating electrode, through communication link 13′. Because the electrical stimulation is largely unnoticeable to the patient, it may be preferred to initially initiate the electrical stimulation. If the electrical stimulation is unsuccessful in perturbing the patient's propensity for seizure, the system 10 may then allow the patient to access the pharmacological agent.

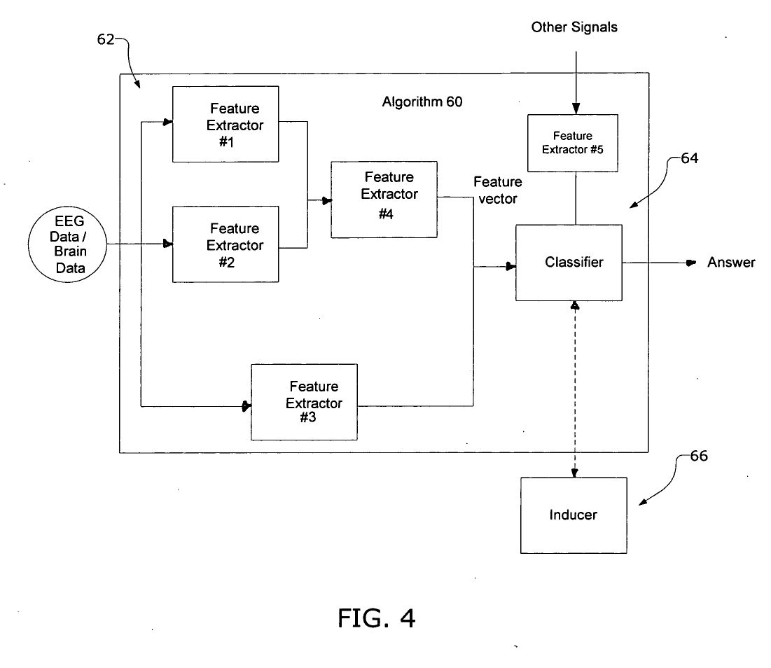

FIG. 3 illustrates one embodiment of a simplified device assembly 12 that is encompassed by the present invention. Device assembly 12 typically carries out the methods of the present invention through a combination of dedicated hardware components, software components, and firmware components. However, if desired, it may be possible to use only software, only hardware, or only firmware. In the illustrated embodiment, device assembly 12 comprises dedicated signal processing hardware 27 (e.g., ASIC (Application Specific Integrated Circuit), FPGA (Field Programmable Gate Array), DSP (Digital Signal Processor), or the like), one or more processors 28, and one or more memory modules 30 that are in communication through a system bus 32. System bus 32 may be analog, digital, or a combination thereof, and system bus 32 may be wired, wireless, or a combination thereof. For ease of reference system bus 32 is illustrated as a single component, but as known to those of skill in the art, system bus 32 will typically comprise a separate data bus and power bus.

Memory 30 may be used to store some or all of the constructs of the software algorithms and other software modules that carry out at least some of the functionality of the present invention. Memory 30 may also be used to record the patient's neural state, propensity for seizure, store data regarding communications provided to the patient, store data regarding inputs received from the patient, store some or all of the raw data measured by sensors, store filter settings, store control law gains and parameters, store therapeutic treatments, protocols, or clinician recommendations, or the like. While processor 28 and memory 30 are illustrated as a single element, it should be appreciated that the processor 28 and memory 30 may take the form of a plurality of different memory modules, in which various memory modules (RAM, ROM, EEPROM, volatile memory, non-volatile memory, or any combination thereof) are in communication with at least one of the processors 28 to carry out the present invention.

A system monitor 33 may be coupled to system bus 32. System monitor 33 is configured to monitor and automatically stop or otherwise interrupt processor 28 and provide some sort of notification to the patient in the event that the power source in device assembly 12 has failed or is about to fail, or if another error in device assembly 12 has occurred. Furthermore, system monitor 33 may be coupled to a reed switch (not shown) or other means that allow the patient to manually actuate system monitor 33 so as to stop or start delivery of therapy or to otherwise actuate or stop system 10. Typically, the patient may activate the reed switch with an external magnet or wand (not shown).

Optionally, system monitor 33 may be in communication with an output assembly 35 via system bus 32. Output assembly 35 may comprise a vibratory mechanism, an acoustic mechanism, a shock mechanism, or the like. System monitor 33 may automatically actuate output assembly 35 to deliver a vibratory signal, audio signal, or electrical shock to indicate to the patient that there an error in device assembly 12 or maintenance is needed to the system 10. Advantageously, the output from output assembly 35 may itself be useful for preventing the neurological event from occurring (e.g., prevent the predicted epileptic seizure).

Processor 28 may be coupled to a system clock 36 for timing and synchronizing the system 10. System clock 36 or additional clocks, such as system monitor clock 36′ may also provide timing information for system monitor 33, or for providing timing information related to therapy delivery, recorded neural state measurements, delivery of instructions to the patient, response by the patient, time stamping of inputs from patients, or the like.

Device assembly 12 may comprise a rechargeable or non-rechargeable power source 37. Some examples of a power source that may be used with the device assembly 12 include the batteries of the type that are used to power a heart pacemaker, heart defibrillator, or neurostimulator. Power source 37 provides power to the components of device assembly 12 through system bus 32. If the power source is rechargeable, a recharging communication interface, such as recharging circuitry 38 will be coupled to power source 37 to receive energy from an external recharging assembly (not shown), such as an RF transmitter or other electromagnetic field, magnetic field, or optical transmission assembly. Such recharging assemblies are commonly used with rechargeable neurostimulators and are well known to those of ordinary skill in the art.

In addition to the recharging communication interface 38, device assembly 12 will typically comprise one or more additional communication interfaces for communicating with patient interface assembly 14, pharmacological agent dispenser 16, patient communication assembly 16, and/or the clinician communication assembly. For example, device assembly 12 may comprise a signal conditioning assembly 40 that acts as an interface between the patient interface assembly 14 and device assembly 12. Signal conditioning assembly 40 which may be comprised of hardware, software, or both, may be configured to condition or otherwise pre-process the raw signals (e.g., EEG data, ECG data, temperature data, blood flow data, chemical concentration data, etc.) received from patient interface assembly 14. Signal conditioning assembly 40 may comprise any number of conventional components such as an amplifier, one or more filters (e.g., low pass, high pass, band pass, notch filter, or a combination thereof), analog-to-digital converter, spike counters, zero crossing counters, impedance check circuitry, and the like.

Device assembly 12 may also comprise a therapy assembly 42 to interface with patient interface assembly 14′, patient communication assembly 18, and/or dispenser 16. Therapy assembly 42 may be comprised of software, hardware, or both, and may receive the output from processor 28 (which may be the yes/no prediction of an onset of a seizure in a near term, propensity for a future seizure, probability output of a seizure, estimated time horizon to a predicted seizure, the patient's measured neural state, a signal that is indicative of the patient's neural state, a control signal for controlling the therapy assembly, or the like) and use the output to generate or modify the therapy and/or communication that is delivered to the patient. The therapy assembly may include a therapy algorithm, a control circuit and associated software, an output stage circuit, and any actuators including pulse generators, patient interfaces, electrode interfaces, drug dispenser interfaces, and other modules that indicate a preventative or therapeutic action to be taken by or on behalf of the patient.

One or more communication interfaces 44 may be used to facilitate communication between device assembly 12 and a remote clinician communication assembly, patient communication assembly 18, a local personal computer, a network, or pharmacological agent dispenser 16, so as to allow for communication of data, programming commands, patient instructions, control signals, or the like. As noted above, communication may be carried out via conventional wireless protocols, such as telemetry, inductive coil links, RF links, other electromagnetic links, magnetic links, infrared links, optical links, ultrasound links, or the like. Communication interface 44 will typically include both a receiver and a transmitter to allow for two-way communication so as to allow for providing software updates to device assembly 12, transmit stored or real-time data (e.g., neural state data, other processed physiological data, raw data from sensors, etc.) to the patient/clinician communication assembly, transmit inputs from the patient/clinician, or the like. However, if only one-way communication is desired, then communication interface 44 will include only one of the receiver and transmitter.

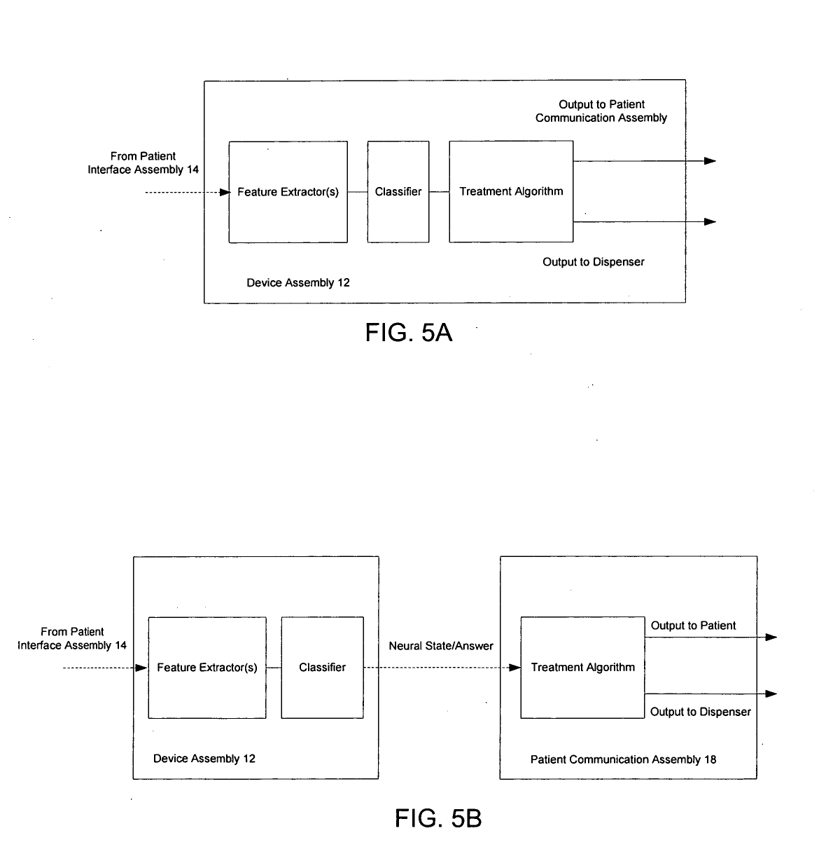

FIG. 4 illustrates one embodiment of a predictive algorithm 60 that may be used by the system 10 of the present invention to characterize the patient's propensity for a future seizure. Predictive algorithms 60 are routinely considered to be comprised of arrangements of feature extractors or measures 62, and classifiers 64. Feature extractors 62 are used to quantify or characterize certain aspects of the measured input signals, which may be any patient dependent parameter. In one preferred embodiment, the predictive algorithm 60 comprises feature extractors for brain signals (e.g., EEG signals, brain temperature signals, brain blood flow, etc.) that are used to characterize the patient's neural state and feature extractors for other patient parameters (e.g., non-brain, physiological signals, patient history, patient inputs). The predictive algorithm typically uses some combination of the brain signal and other patient parameters to characterize the patient's propensity for seizure, but it may be possible that only the brain signals (e.g., neural state) or only the other patient parameters may be used to characterize the patient's propensity for seizure.

One or more classifiers 64 are then used to combine the results obtained from the feature extractors into an overall answer or result. The classifier 64 may be customized for the individual patient and the classifier may be adapted to use only a subset of the features that are most useful for the specific patients. Additionally, over time, as the system adapts to the patient, the classifier 64 may reselect the features that are used for the specific patient.

In use, signals from patient interface assembly 14 may be transmitted to predictive algorithm 60. The signals may be first pre-processed by the signal conditioning assembly (not shown). The signals from patient interface assembly 14 typically include at least one signal from the brain (e.g., EEG signal), and preferably a plurality of EEG signals from an intracranial electrode array. Feature extractors 62 receive the signals and extract various quantifiable features or parameters from the signal to generate an output for classifier 64. Feature extractor 62 may extract univariate and bivariate measures and may use linear or non-linear approaches. While the output from feature extractor 62 may be a scalar, the output is typically in the form of a multivariable feature vector. As shown in FIG. 4, each of the features themselves may be combined with other features and used as inputs for a separate feature extractor. For example, in the illustrated example, the output from Feature Extractor #1 and the output from Feature Extractor #2 are used as inputs into Feature Extractor #4. Any number of different feature extractors may be used to extract features from the signals from the patient's brain to characterize the patient's neural state. Different combinations of features and/or different features themselves may be used for different patients to characterize the patient's neural state.

Some examples of potentially useful features to extract from the signals for use in determining the patient's neural state, include but are not limited to, brain temperature, blood flow in the brain, alpha band power (8-13 Hz), beta band power (13-18 Hz), delta band power (0.1-4 Hz), theta band power (4-8 Hz), low beta band power (12-15 Hz), mid-beta band power (15-18 Hz), high beta band power (18-30 Hz), gamma band power (30-48 Hz), second, third and fourth statistical moment of the EEG amplitudes, spectral edge frequency, decorrelation time, Hjorth mobility (HM), Hjorth complexity (HC), the largest Lyapunov exponent L(max), effective correlation dimension, local flow, entropy, loss of recurrence LR as a measure of non-stationarity, mean phase coherence, conditional probability, brain dynamics (synchronization or desynchronization of neural activity, T-index, angular frequency, and entropy), line length calculations, area under the curve, first, second and higher derivatives, integrals, or a combination thereof. Some additional features that may be useful are described in Mormann et al., “On the predictability of epileptic seizures,” Clinical Neurophysiology 116 (200) 569-587.

In addition to the neural state features, algorithm 60 may also comprise feature extractors to extract features from other patient dependent parameters, such as other physiological signals (e.g., electromyography (EMG) signals, electrocardiogram (ECG) signals, temperature signals other portions of the body, blood flow measurements from other parts of the body, heart rate signals and/or change in heart rate signals, respiratory rate signals and/or change in respiratory rate signals, chemical concentrations of AED or other medications, pH in the blood, or other portions of the body, blood pressure, or other vital signs or physiological parameters of the patient's body), a patient's history (e.g., seizure patterns), and other patient inputs. For example, inputs from the patient regarding having an aura or prodrome, AED intake, alcohol intake, sleep state, etc., may be useful in characterizing the patient's propensity for a seizure.

At the classifier, the extracted features are combined to form a feature vector (or scalar). The feature vector is classified to provide a logical answer or weighted answer. In order to provide the classifications for the classifier 64, an inducer 66 may use historical/training feature vector data to automatically train the classifier 64. The inducer 66 may be used prior to implantation and/or may be used to adaptively monitor the propensity for seizure and dynamically adapt the classifier in vivo. Using any of the accepted classification methods known in the art, the measured feature vector is compared to historical or baseline feature vectors to classify the patient's propensity for the onset of a future epileptic seizure. For example, the classifier may comprise a support vector machine classifier, a predictive neural network, artificial intelligence structures, a k-nearest neighbor classifier, or the like

As it relates to epilepsy, one implementation of the classification of states defined by the classifier could include a “normal” state or inter-ictal state (e.g., “low propensity for a seizure”), an “abnormal” state or pre-seizure state (sometimes referred to herein as “pre-ictal state” or “elevated propensity for a seizure”), a seizure state or ictal state, and a post-seizure state or post ictal state. Since a significant purpose of the algorithm is to determine where the patient's neural state lies on a continuum from “normal” to “abnormal,” it may be desirable to have the classifier classify the patient's neural state as being in one of the two most important states—pre-ictal state or inter-ictal state. In this case, a patient would be advised of the neural state having transitioned or deviating from an inter-ictal state to a pre-ictal state.

As noted above, instead of providing a logical answer, it may be desirable to provide a weighted answer so as to further delineate within the pre-ictal state to further allow system 10 to provide a more specific output communication for the patient and/or dispenser 16. For example, instead of a simple logical answer (e.g., pre-ictal or inter-ictal) it may be desirable to provide a weighted output in the form of a simplified neural state index (NSI) that quantifies the patient's propensity using some predetermined scale (e.g., scale of 1-10, with a “1” meaning “normal” and a “10” meaning seizure is imminent). For example, if it is determined that the patient has entered the pre-ictal state, but the seizure is likely to occur on a long time horizon, the output signal could be weighted to be reflective of the long time horizon, e.g., an NSI output of “5”. However, if the neural state indicates that the patient is in a pre-ictal state and it is determined that the seizure is imminent within the next 10 minutes, the output could be weighted to be reflective of the shorter time horizon to the seizure, e.g., an NSI output of “9.” On the other hand, if the patient's neural state is normal, the algorithm may provide an NSI output of “1”. Another implementation involves expressing the inter-ictal and pre-ictal states as a continuum, with a scalar or vector of parameters describing the actual state and its variation. Such a continuous variable or set of variables can be communicated to the patient, enabling the patient to employ his or her own judgment and interpretation to then guide palliative or preventative behaviors or therapies.

Once the classifier has classified the patient's propensity for seizure, the output of the classifier (e.g., elevated propensity or low propensity, graded propensity, NSI, etc.) is transmitted to the treatment algorithm, such as a configurable communication state machine, where the appropriate action is determined.

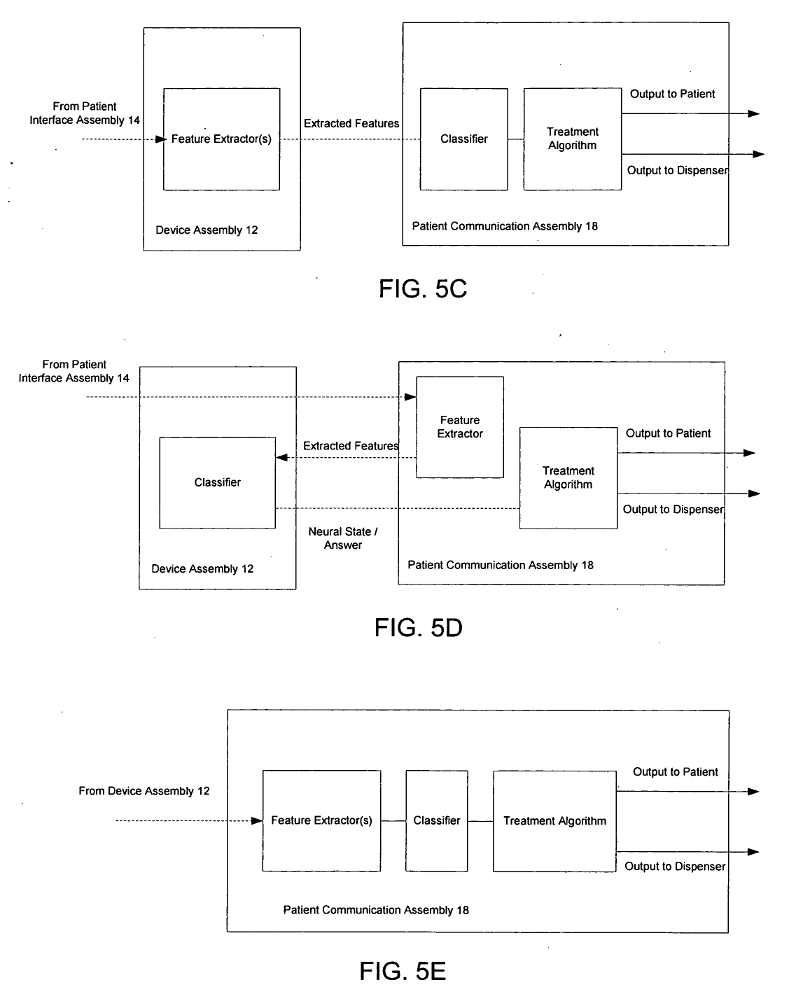

Depending on the desired characteristics of system 10, the predictive algorithms and treatment algorithms may be embodied in one or more components of system 10 and may be implanted in the patient's body, external to the patient's body, or a combination thereof. FIGS. 5A to 5E illustrate a number of different embodiments of how processing may be carried out using the predictive algorithm 60 and therapy algorithms of the present invention.

As illustrated in FIG. 5A in one embodiment both the predictive algorithm and treatment algorithm may be processed in the device assembly 12 that is implanted in the patient's body. The feature extractor and classifier may be used to characterize the patient's propensity for a future seizure. The treatment algorithm may receive the characterization and generate the communication outputs as described above, and transmit the communication outputs transcutaneously to at least one of a patient communication assembly 18 and/or dispenser 16.

In the embodiment illustrated in FIG. 5B, the predictive algorithm (e.g., feature extractor and classifier) may be embodied in implanted device assembly 12 while the treatment algorithm may be embodied in an external patient communication assembly 18. In such embodiments, the patient's propensity for seizure, or whatever output is generated by the predictive algorithm that characterizes the patient's propensity for the onset of the future seizure (shown as “Answer”) is transmitted wirelessly through the patient's skin to the external patient communication assembly 18, where the treatment algorithm receives the output from the predictive algorithm and uses the data to determine an appropriate therapy and generate the communication outputs to the patient and/or dispenser 16. Such embodiments have the benefit of sharing processing power, while reducing the battery usage of the implanted assembly 12, thus prolonging the life of device assembly 12. Additionally, because the treatment algorithm is external to the patient's body, reprogramming or updating may be performed more easily, and reprogramming of the implanted component (which is more difficult) is not required.

In yet another embodiment of the present invention, as shown in FIG. 5C it may be possible to perform some of the processing of the signals in the implanted device assembly 12 and some of the processing of the signals and treatment determination in an external device, such as the patient communication assembly 18. For example, a first level of processing may extract one or more features from the one or more signals with feature extractors that are in the implanted device assembly 12. Selected extracted features may be transcutaneously transmitted to the patient communication assembly, where a second level of processing may be performed on the extracted features, such as classifying the features to characterize the patient's propensity for the onset of a future seizure. Thereafter, an appropriate action (if needed) may be determined by the treatment algorithm and output to the patient and/or dispenser 16. While the treatment algorithm is illustrated as being in the patient communication assembly 18, it should be appreciated that the treatment algorithm may be embodied in either the implanted device assembly 12 or the external patient communication assembly 18. Advantageously, since most of the decision making is done in a device that is external to the patient's body, such embodiments may require less computing power in the implanted device assembly 12, thus prolonging the battery life of the implanted device assembly. Because less data is transmitted from the implanted device assembly 12 to the external patient communication assembly 18, such configurations may reduce the bandwidth requirements for the telemetered data. Furthermore, because the classifier and treatment algorithm (e.g., the prediction and judgment algorithms) are embodied in an external assembly, such an embodiment will be easier for the clinician to reprogram.

As shown in FIG. 5D, in a variation to the embodiment of FIG. 5C, it may be possible to switch the positions of the classifier and the feature extractors so that a first level of processing may be performed external to the body. Pre-processed signals (e.g., filtered, amplified, conversion to a digital signal) may be transcutaneously transmitted from device assembly 12 to the patient communication assembly 18 where one or more features are extracted from the one or more signals with feature extractors. Some or all of the extracted features may be transcutaneously transmitted back into the device assembly 12, where a second level of processing may be performed on the features, such as classifying the features and other signals to characterize the patient's propensity for the onset of a future seizure. Thereafter, the patient's propensity for seizure or other answer may be transmitted to the treatment algorithm (which may be in the device assembly 12 or the patient communication assembly 18) to determine an appropriate action (if needed). If desired, to improve bandwidth, the classifier may be adapted to allow for transmission or receipt of only the features from the patient communication assembly that are predictive for that individual patient. Advantageously, because feature extractors may be computationally expensive and power hungry, it may be desirable to have the feature extractors external to the body, where it is easier to provide more processing and larger power sources.

In yet another embodiment shown in FIG. 5E, it may be possible that most or all of the processing of the signals measured by patient interface 14 is done in the patient communication assembly 18 that is external to the patient's body. In such embodiments, the implanted device assembly 12 would receive the signals from patient interface 14 and may or may not pre-process the signals and transcutaneously transmit some or all of the raw neural data or a processed or compressed form of the neural data to the external patient communication assembly 18, where the characterization of the patient's propensity for the future seizure and therapy determination is made. Advantageously, such embodiments may allow for greater processing power that may be needed to implement complex algorithms. Furthermore, such a configuration may facilitate easier customization for a given patient and easier reprogramming and retraining of the algorithm.

While FIGS. 4 to 5E illustrate exemplary predictive algorithms of the present invention, a variety of other predictive algorithms may be useful with the systems 10 of the present invention to characterize the patient's propensity for the future seizure. Some examples of other useful detection or prediction algorithms include those described in U.S. Pat. No. 3,863,625 to Viglione, U.S. Pat. No. 6,658,287 to Litt, U.S. Pat. No. 5,857,978 to Hively, and U.S. Pat. No. 6,304,775 to Iasemidis, U.S. Pat. No. 6,507,754 to Le Van Quyen et al., U.S. Pat. No. 6,594,524 to Esteller et al. Any of such detection and prediction algorithms may be used by system 10 of the present invention to produce an output that may be used by the treatment algorithm to determine the communication (e.g., recommendation, instruction, or warning) that is output to the patient and dispenser 16. For example, one or more probability outputs or time horizons of Litt's '978 algorithm may be used to determine the appropriate action output that is provided to the patient and may be used as inputs to determine the propensity for a seizure.

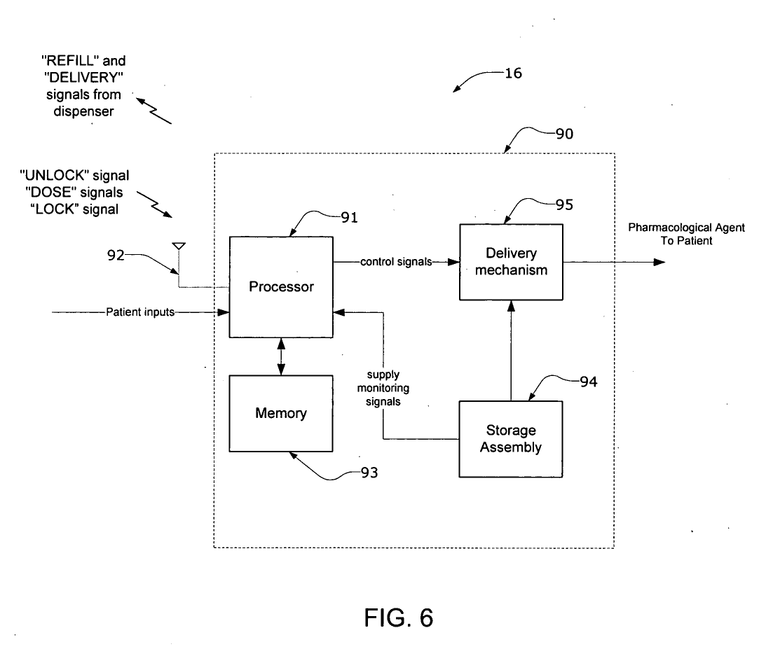

FIG. 6 illustrates one embodiment of a simplified pharmacological agent dispenser 16 of the present invention. The pharmacological agent dispenser 16 typically comprises a housing 90. A processor 91 is disposed within housing 90 and is in communication with a communication port 92 that provides wireless or wired communication with at least one of the patient communication assembly 18 and the device assembly 12. A memory 93 may be in communication with processor 91 and typically stores data relating to the usage of dispenser 16. A storage assembly 94 stores the pharmacological agent and is in communication with a delivery mechanism 95 and the processor 91. Control signals from processor 91 to delivery mechanism 95 may toggle the delivery mechanism between a “locked state” and an “unlocked state”. The control signals may also be used to specify the dosage that the patient is allowed to administer.

In use, when it is determined that the patient is at an elevated propensity for a future seizure, a signal is transmitted from patient communication assembly 18 or device assembly 12 that instructs processor 91 to unlock the dispenser. Optionally, a “Dose” signal may also be transmitted to processor 91 that indicates an appropriate dosing of the pharmacological agent. Communication port 92 receives the “Unlock” signal and/or “Dose” signal and transmits such signals to processor 91, which in turn may deliver one or more control signals to delivery mechanism 95 to unlock the dispenser to allow the user to access the pharmacological agent and to set the appropriate dosage that can be administered.

Delivery mechanism 95 controls the administration of the pharmacological agent. Delivery mechanism typically includes a release mechanism (e.g., valve, nozzle, needle, door, etc.) and some sort of locking mechanism that prevents activation of the release mechanism and access to the pharmacological agent. Typically, the locking mechanism is maintained in a locked state to prevent the patient from activating the release mechanism. When the control signal is received from the processor 91, the locking mechanism moves to an unlocked state to allow the patient to access the pharmacological agent. There are a number of different delivery mechanisms and release mechanisms known to those of ordinary skill in the art which may be used with this invention.

Storage assembly 94 may be in the form of any conventional drug container, including but not limited to, a canister, reservoir, catheter, pill box, syringe body, or other conventional storage assemblies. The storage assembly may include means that are able to monitor the supply of pharmacological agent that is remaining and deliver a “Supply” signal to the processor 91 that is indicative of the amount of pharmacological agent remaining. In other embodiments, the processor 91 may be configured to automatically monitor the supply of pharmacological agent by monitoring the number of times that the pharmacological agent has been accessed, the number of times that the delivery mechanism has been activated or the like. In any of the embodiments, if it is determined that the supply in storage assembly 94 is low, the processor 91 may transmit a “Refill” signal through communication port 92 back to device assembly 12 or patient communication assembly 18 to inform the patient of the low supply of pharmacological agent.

Memory 93 is in communication with processor 91 and may be used to store the timing and dosage history of the patient's usage. For example, every time the delivery mechanism 95 is activated to dispense the pharmacological agent (shown in FIG. 6 as “Patient Inputs”) a “Delivery” signal may be generated and sent to processor 91. Processor 91 may time stamp the “Delivery” signal and store it in memory 93 and/or transmit the “Delivery” signal to device assembly 12 and/or patient interface assembly 18 so as to inform device assembly 12 and/or patient communication assembly 18 that the pharmacological agent was administered. Additionally, memory 93 may also store data regarding the times that the delivery mechanism 95 was unlocked but the patient didn't administer the pharmacological agent. Any of such data may be downloaded to the patient communication assembly 18 and/or device assembly 12 for future analysis. Alternatively, a clinician may download such information directly from memory 93 during an office visit. Such data may be used to monitor both patient compliance and abuse of the pharmacological agent.

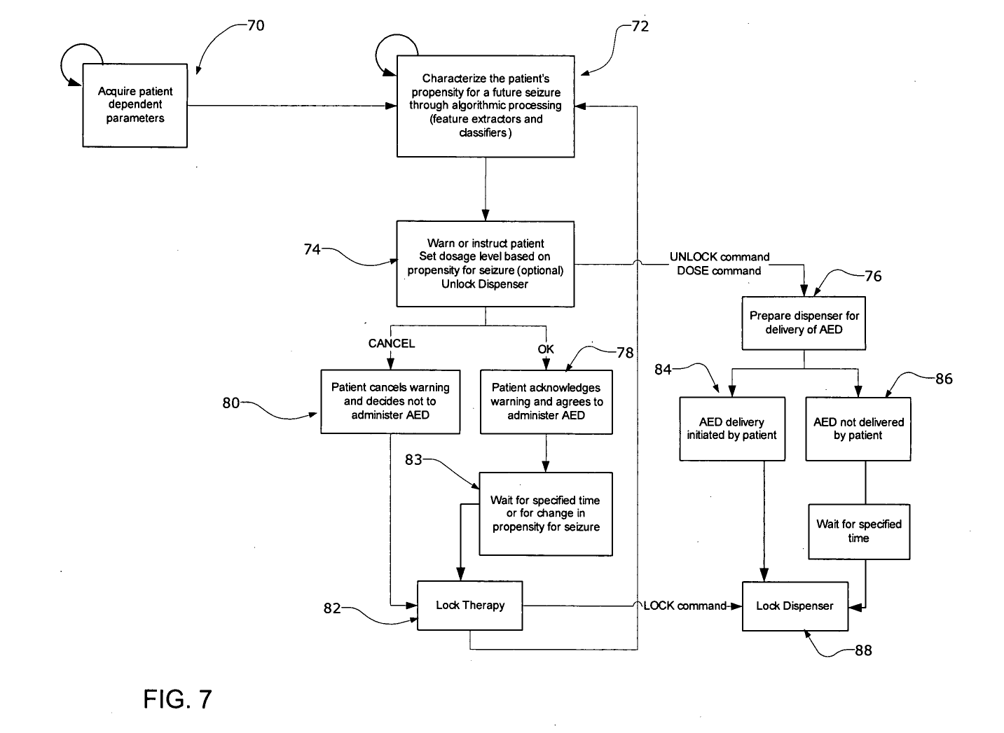

FIG. 7 illustrates a method that is encompassed by the present invention. The pharmacological agent dispensers of the present invention will typically be maintained in a locked state so as to prevent misuse of the pharmacological agent stored in the dispenser. The patient interface assembly is used to measure one or more patient dependent parameters (Step 70). The patient dependent parameters (e.g., EEG) that are indicative of the patient's propensity for a future seizure, are pre-processed (e.g., filtered, converted to a digital signal, amplified, etc.) and then input into the predictive algorithm, where one or more features are extracted from the signal. In the embodiment of FIG. 7, the filtering and feature extraction are performed in the implanted device assembly, and such data is transmitted wirelessly to a device that is external to the patient's body, where higher level algorithmic processing is performed (e.g., high level feature extraction and classification) to characterize the patient's neural state and propensity for a future seizure (Step 72). But as can be appreciated, any of the embodiments illustrated in FIGS. 5A to 5E may be used to carry out the methods of the invention.

Once the patient's propensity for seizure is determined by the predictive algorithm, a signal that is indicative of the patient's propensity for seizure is transmitted to a treatment algorithm where it is determined if any action is needed. If an action is needed (e.g., the patient has an increased propensity for a seizure), the appropriate action is determined by the treatment algorithm (such as a digital state machine).

A communication output may be transmitted to the pharmacological agent dispenser 16 and to the patient via the patient communication assembly (Step 74). In the simplest embodiment, the predictive algorithm provides an output that indicates that the patient is trending toward a seizure. In such embodiments, the communication output to the patient may simply be a warning or a recommendation to the patient that was programmed into the system by the clinician. In other embodiments, the predictive algorithm may output a graded likelihood or propensity characterization, a quantitative probability of the onset of the seizure, a time horizon until the predicted seizure will occur, or some combination thereof. In such embodiments, the communication output to the patient may provide a recommendation or instruction that is a function of the likelihood assessment, probability, or time horizon.

In some embodiments, based on the patient's measured propensity, the treatment algorithm may select a dosage level, form, formulation, route of administration of the pharmacological agent. A minimal or otherwise small dosages of agent (e.g., less than a “normal” dosage) may initially be delivered if the patient's propensity for a seizure is only slightly increased, a probability of a seizure is estimated to be low, a long time horizon for a seizure is estimated, and/or the neural state is indicative of a lower likelihood of a seizure. Advantageously, such a small dosage may be able to prevent the onset of the seizure, while reducing and potentially avoiding the deleterious side effects from the agents. Moreover, even if the small dosage of the agent is ineffective, because the propensity for the seizure is low (and the time horizon is long), there will likely be time to iterate through additional dosages of the agent.

A larger dosage may be recommended by the clinician if the patient's initial characterization of the patient's propensity for a future seizure is high, a probability of a seizure is estimated to be high, a short time horizon for a seizure is estimated, and/or the neural state is indicative of a higher likelihood of a seizure. Additionally, the larger dosages may also be recommended when it is determined that initial smaller dosages of the agent were ineffective in perturbing the patient's propensity for seizure back to a normal state. Because the systems and methods of the present invention are able to substantially continuously monitor the patient's propensity for seizure and assess the effect of the initial dosages, the systems of the present invention are able to “learn” the appropriate dosages, forms, formulations of the agents that are effective for the patient. Once the appropriate parameters of the treatment are determined, it may be desirable to recommend the appropriate treatment parameters in future recommendations.

At some point after the predictive algorithm characterizes an increased propensity for a future seizure, one or more communication signals may be transmitted wirelessly to dispenser 16 so as to allow the patient to access the pharmacological agent and/or to set the dosage of the pharmacological agent (Step 76). Allowing the patient to access the pharmacological agent may be carried out after the patient acknowledges the instruction, or the patient may be provided access automatically after or concurrently with the communication to the patient. Allowing the patient to access the AED will typically move the drug dispenser from the locked state to an unlocked state so as to enable the patient to administer the pharmacological agent. In some embodiments, the communication signal to the dispenser may adapt the dispenser to deliver only the desired dosage of the pharmacological agent. For example, if the dispenser is a metered dose inhaler, the signal may reconfigure the dispenser to allow a single bolus to deliver all of the desired agent in one actuation (e.g., 20 μg in a single dosage). In embodiments which do not reconfigure the dosage delivered with each bolus, the present invention may alternatively limit the number of times the dispenser may be actuated, up to the recommended dosage (e.g., two dosages of 10 μg).

After the dispenser is made accessible to the patient, the patient may take the AED (Step 86) or decide to not take the AED (Step 84). If the patient takes the AED, the dispenser will be re-locked to prevent overuse of the dispenser. If the patient does not take the AED, after a specified time the dispenser will be re-locked (Step 88)

The seizure predictive algorithm may continue to characterize the patient's propensity for seizure, and if the elevated propensity for a seizure persists, follow up warnings or recommendations may be provided to the patient and the dispenser could be unlocked again for delivery of additional doses. Depending on the patient's subsequent characterizations of the propensity for seizure, the additional dosages may be maintained at the same level, decreased, increased, or additional parameters may be varied.

As illustrated in FIG. 7, some embodiments the present invention may provide an interactive communication protocol with the patient to improve the monitoring of patient compliance with the recommendations and/or monitor the patient's pharmacological agent intake and the response thereof. The patient may be prompted to acknowledge that the patient received the warning or recommendation or acknowledge that the pharmacological agent has been administered (Step 78). After a specified time or once it is determined that the expected reduction in the patient's propensity has been achieved, a lock therapy signal may be transmitted to the dispenser 16 to prevent misuse of the dispenser (Step 83). However, if the patient chooses not to administer the pharmacological agent, the patient may activate the “cancel button” or a similar input, to indicate to system 10 that the agent was not administered (Step 80) and a lock command may be transmitted to the dispenser 16 to again prevent access to the pharmacological agent (Step 82).

The acknowledgement or cancel input by the patient may be stored in a memory of system and used in subsequent seizure predictions and/or by the clinician in follow-up examinations with the patient. For example, if the patient acknowledges that the pharmacological agent was taken, but the propensity for seizure was not perturbed as expected, such data may indicate that the patient did not actually administer the pharmacological agent or that the pharmacological agent was ineffective in perturbing the patient's propensity for seizure. In either case, the missed or ineffective dosage may be logged into memory. For subsequent recommendations, using such data, the system 10 may provide a follow up communication that either reminds the patient to administer the same dosage of the agent, administer a larger dosage of the same agent, or administer a different pharmacological agent.

In addition to acknowledging the warning or intake of the agent, the patient communication assembly may be used to indicate to system 10 that the patient is having an aura (part of Step 70). Some patients experience an aura in advance of the onset of a seizure. When the patient experiences such an aura, the patient may activate an input on the patient communication assembly, and the “aura input” may be logged in memory and used by the classifiers in future processing to improve the characterizations of the patient's propensity for seizure. All of such outputs and inputs from the patient may be stored in a memory and later analyzed by the clinician and used by the predictive algorithm to characterize the patient's propensity for a seizure.

The systems and methods of the present invention may also be used to monitor and document the patient's administration of the pharmacological agent. Such information could be useful for the training of the seizure detection/prediction algorithms as well as characterizing the effectiveness of the AEDs for the patient. The present invention may monitor the patient's administration of the pharmacological agent in a number of ways. For example, system 10 may track the patient's rate of access or number of times that that pharmacological agent dispenser was made accessible to the patient (without any actual knowledge as to whether or not the patient actually administered the drug). Similarly, tracking administration of the pharmacological agent may also be carried out by storing the number of times that the patient acknowledges that the pharmacological agent has been administered. In other embodiments, every time the dispenser 16 is actually activated, a signal may be generated and stored in a memory of system 10. For example, every time the metered dose inhaler is actuated by the patient or every time a pill dispenser door is opened, a signal may be sent to system 10 that indicates that the dispenser 16 was activated, thus assessing compliance. If the metered dose inhaler or pill dispenser is able to adjust the dosage of the agent that is dispensed, the signal may also include dosage information.

In other embodiments, it may be possible to substantially continuously characterize the propensity for seizure, and whenever the patient's propensity for seizure is perturbed after the recommendation or warning, it may be assumed that the pharmacological agent has been administered. If the patient's propensity for seizure is not perturbed after a recommendation to administer an agent, such a non-perturbation of the propensity for seizure may also be stored in memory. In yet another embodiment, it may be possible that the patient interface assembly 14 of system 10 may include an appropriately placed biochemical sensor that measures the plasma level of the pharmacological agent or its derivatives, precursors, or metabolites in the patient's blood stream. Signals from the sensor that are indicative of the plasma level of the pharmacological agent may be used to monitor the patient's intake of the pharmacological agent.

System 10 may use one or more of the above methods of monitoring the administration of the pharmacological agent as a safeguard to prevent an overdose. For example, in one embodiment a maximum threshold of medication over a period of time may be set by the clinician, and the maximum threshold may be saved in a memory of system 10. If the maximum threshold of medication is reached for the predetermined period of time (e.g., day, week, month, year, or other predetermined time period), system 10 will be prevented from communicating an instruction to the patient to take additional dosages of the prescribed pharmacological agent. Instead of providing the standard instruction, system 10 may be configured to provide a warning to the patient to indicate that the maximum amount of medication is being reached. Such a warning would allow the patient to contact their clinician or the like. In such cases, it may be desirable to have the clinician program a second, alternative pharmacological agent or other appropriate action into memory that would then be output to the patient.

It may be possible to configure system 10 so that when the amount of medication taken approaches the maximum, a signal may be sent to a server that the clinician may access or a signal may be sent directly to a clinician communication assembly that is in communication with system 10 so as to warn the clinician of the patient's status. In some embodiments, system 10 may be configured to regularly communicate pharmacological agent updates and/or neural state history updates (e.g., number of seizures) to the clinician. This has considerable value in assessing and preventing the occurrence of an overdose of antiepileptic and other drugs with potentially significant side effects, including benzodiazepines or barbiturates.

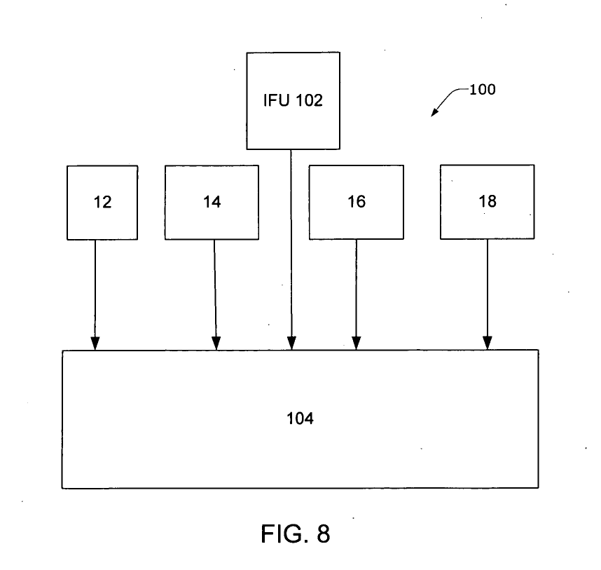

Referring now to FIG. 8, the present invention will further comprise kits 100 including any combination of the components described above, instructions for use (IFU) 102, and packages 104. Typically, the kit 100 will include some combination of the device assembly 12, one or more patient interface assemblies 14, a pharmacological agent dispenser 16, and patient communication assembly 18. The IFU 102 will set forth any of the methods described above. Package 104 may be any conventional medical device packaging, including pouches, trays, boxes, tubes, or the like. The instructions for use 102 will usually be printed on a separate piece of paper, but may also be printed in whole or in part on a portion of the packaging 104.

The AEDs that may be administered by the present invention include, but are not limited to, Hydantoins, Anti-seizure Barbiturates, Deoxybarbiturates, Iminostilbenes, Succinimides, Valproic Acid, Benzodiazepines, Lamotrigine, Levetiracetam, Tiagabine, Topiramate, Zonisamide, and Vigabatrin. The form of the AED will vary depending on the type of dispenser used, however, the AEDs may be delivered in any form known in the art e.g., pills, liquid, powder, aerosol, cream, suppository, or the like. A more detailed description of the dosages, forms, formulations and routes of administrations of AEDs that may be administered using the dispensers 16 of the present invention were previously described in commonly owned, copending U.S. patent application Ser. No. ______, filed Dec. 28, 2005, entitled “Methods and Systems for Recommending an Appropriate Action to a Patient for Managing Epilepsy and Other Neurological Disorders,” (BNC Docket No. 2.00US; WSGR Docket No. 31685-713.201) and U.S. patent application Ser. No. ______, filed Dec. 28, 2005, entitled “Methods and Systems for Recommending a Pharmacological Treatment to a Patient for Managing Epilepsy and Other Neurological Disorders” (BNC Docket No. 2.01US; WSGR Docket No. 31685-713.202), both to Leyde et al.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. For example, the systems of the present invention could be replaced by a more generic computing platform such as a PDA, cellular phone, or other handheld consumer electronic, medical or custom device. The device could run software that is programmed by a clinician to only unlock the dispenser 16 at a defined interval and provide documentation as to the actual delivery and dosage schedule of the drug delivery system. This system could apply to any patient-worn or patient carried device that dispenses medication.

Furthermore, instead of using the systems of the present invention for acute dosages of AEDs, the present invention may be used to administer a modified or altered chronic regimen of AEDs. Such a regimen could potentially prescribe lower dosages of AEDs than currently used, and when the system determines that the patient has an increased propensity for a future seizure, the system could modify or alter the scheduling and dosing of a chronically prescribed pharmacological agent, such as an AED, to optimize or custom tailor the dosing to a particular patient at a particular point in time. This allows for improved (1) efficacy for individual patients, since there is variation of therapeutic needs among patients, and for (2) improved response to variation in therapeutic needs for a given patient with time, resulting form normal physiological variations as well as from external and environmental influences, such as stress, sleep deprivation, the presence of flashing lights, alcohol intake, and the like. Consequently, the present invention is able to provide a lower chronic plasma level of the AED and modulate the intake of the prescribed agent in order to decrease side effects and maximize benefit of the AED.

It is intended that the following claims define the scope of the invention and that methods and structures within the scope of these claims and their equivalents be covered thereby.

What is claimed:

1. A method for controlling administration of a pharmacological agent for treating epilepsy, the method comprising processing one or more signals from a patient to characterize a patient's propensity for a future seizure, and based upon measuring an increased propensity for the future seizure, allowing the patient to access the pharmacological agent from a dispenser.

2. The method of claim 1 wherein processing the one or more signals comprises:
extracting features from the one or more signals with a feature extractor;
transmitting at least some of the extracted features to a classifier; and
classifying at least some of the transmitted features to characterize the patient's propensity for the future seizure.

3. The method of claim 2 wherein the features comprise a mathematical feature derived from a physiological signal from the patient.

4. The method of claim 2 wherein the patient's propensity for a future seizure is indicated by a prediction interval, wherein the prediction interval is at least about 30 seconds.

5. The method of claim 2 wherein the feature extractor is embodied within a device that is implanted in the patient's body and the classifier is embodied in a device that is external to the patient's body.

6. The method of claim 2 wherein the feature extractor and classifier are embodied within a device that is implanted in the patient's body.

7. The method of claim 2 wherein the feature extractor and the classifier are embodied in a device that is external to the patient's body.

8. The method of claim 2 wherein the classifier is embodied within a device that is implanted in the patient's body and the feature extractors are embodied in a device that is external to the patient's body.

9. The method of claim 2 further comprising transmitting selected signals to the classifier, wherein classifying comprises classifying at least some of the transmitted features and the selected signals to characterize the patient's propensity for the future seizure.

10. The method of claim 1 wherein processing the one or more signals to characterize the patient's propensity for the future seizure comprises characterizing a patient's neural state.

11. The method of claim 10 wherein allowing the patient to access the pharmacological agent from the dispenser comprises allowing administration of only an amount of pharmacological agent that is a function of the patient's characterized neural state.

12. The method of claim 1 wherein allowing the patient to access the pharmacological agent comprises unlocking the dispenser.

13. The method of claim 1 further comprising monitoring the patient's access to the pharmacological agent from the dispenser.

14. The method of claim 13 further comprising limiting the patient's access to the pharmacological agent when the access to the pharmacological agent reaches a threshold.

15. The method of claim 13 wherein monitoring the patient's access to the pharmacological agent comprises measuring a number of times the pharmacological agent was made accessible over a time period.

16. The method of claim 13 wherein monitoring the patient's access to the pharmacological agent comprises measuring a number of times the pharmacological agent dispenser was activated.

17. The method of claim 13 wherein monitoring the patient's access to the pharmacological agent comprises a number of times the patient affirmed that the pharmacological agent was taken.

18. The method of claim 13 wherein monitoring the patient's access to the pharmacological agent comprises monitoring the one or more signals from the patient for an expected response to the pharmacological agent.

19. The method of claim 1 further comprising delivering a communication to the patient that is indicative of the patient's increased propensity for the future seizure.

20. The method of claim 19 wherein the communication to the patient comprises a recommendation to take the pharmacological agent.

21. The method of claim 20 wherein the recommendation to take the pharmacological agent is indicative of at least one of a dosage, form, formulation, and route of administration of the pharmacological agent.

22. The method of claim 1 wherein the one or more signals comprises an input from the patient.

23. The method of claim 22 wherein the input from the patient comprises an affirmation from the patient that the pharmacological agent has been taken.

24. The method of claim 23 comprising using the affirmation from the patient that the pharmacological agent has been take in future characterizations of the patient's propensity for seizures.

25. A method for controlling administration of an anti-epileptic drug, the method comprising:
measuring one or more signals from a patient;
extracting one or more features from the one or more signals;
classifying at least some of the extracted features to characterize the patient's propensity for a future seizure; and
based upon the patient's increased propensity for the future seizure, transmitting a signal to a drug dispenser that enables the patient to dispense the pharmacological agent from the pharmacological agent dispenser.

26. The method of claim 25 further comprising determining an appropriate pharmacological agent therapy for managing the predicted seizure based on the patient's propensity for the future seizure.

27. The method of claim 26 comprising transmitting a communication to the patient that is indicative of at least one of the patient's increased propensity for the future seizure and the appropriate pharmacological agent therapy.

28. The method of claim 26 wherein the communication to the patient is indicative of a suggested dosage of the pharmacological agent, wherein the suggested dosage is a function of the patient's propensity for the future seizure.

29. The method of claim 26 wherein the patient's propensity for the future seizure is a function of at least one of an estimated probability of the future seizure an estimated time horizon to the future seizure.

30. The method of claim 26 wherein the patient's propensity for the future seizure is a function of a patient's neural state.

31. The method of claim 25 wherein the features comprise mathematical features that are derived from a physiological signal from the patient.

32. The method of claim 25 wherein enabling the patient to dispense the pharmacological agent from the dispenser further comprises limiting the amount of pharmacological agent that is dispensed to an amount that corresponds to the patient's propensity for the future seizure.

33. The method of claim 25 wherein enabling the patient to dispense the pharmacological agent from the pharmacological agent dispenser comprises unlocking the dispenser.

34. The method of claim 25 further comprising monitoring the patient's dispensing of the pharmacological agent from the dispenser.

35. The method of claim 34 further comprising limiting the patient's dispensing of the pharmacological agent when the amount of pharmacological agent dispensed reaches a threshold.

36. The method of claim 25 further comprising transmitting selected signals directly to the classifier, wherein classifying comprises classifying at least some of the transmitted features and the selected signals to characterize the patient's propensity for the future seizure.

37. A method for controlling administration of a pharmacological agent for managing a predicted seizure, the method comprising:
characterizing a patient's neural state to estimate a patient's propensity for a future seizure;
wherein if the patient's neural state is indicative of an increased propensity for a future seizure; processing the neural state to determine an appropriate acute pharmacological treatment for managing the future seizure;
providing an output to the patient; and
transmitting a signal to a pharmacological agent dispenser that permits the use of the pharmacological agent dispenser and administration of the appropriate pharmacological treatment to the patient.

38. The method of claim 37 wherein the output to the patient is indicative of the appropriate pharmacological treatment.

39. The method of claim 37 wherein the pharmacological agent dispenser is configured to limit administration to only the amount of pharmacological agent that corresponds to the patient's characterized neural state.

40. The method of claim 37 wherein parameters of the appropriate pharmacological treatment are a function of at least one of the patient's neural state and the patient's propensity for a future seizure.

41. The method of claim 40 wherein the parameters comprise at least one of dosage, form, formulation, and route of administration.

42. A method of communicating with a drug delivery assembly that is external to a patient's body, the method comprising:
measuring one or more physiological parameters with one or more implanted sensors;
transcutaneously transmitting one or more signals from the implanted sensors that are reflective of the measured physiological parameters to a predictive algorithm that is external to the patient's body; and
analyzing the one or more signals with the predictive algorithm to measure a patient's neural state that is indicative of a patient's propensity for an onset of a future seizure,
wherein if the patient's neural state indicates an increased propensity for the onset of a future seizure, transmitting a signal to a manually activatable drug delivery device that is external to the patient's body.

43. A method of predicting an onset of a seizure, the method comprising:
monitoring one or more signals from the patient;
extracting features from the one or more signals in a device that is implanted in the patient's body;
transmitting at least some of the extracted features transcutaneously to a device that is external to the patient's body; and
classifying at least some of the transmitted features to characterize the patient's propensity for a future seizure in the device that is external to the patient's body.

44. The method of claim 43 wherein upon the characterization of an increased propensity for the future seizure, the method comprises enabling access to a pharmacological agent in a dispenser that is external to a patient's body.

45. The method of claim 44 wherein enabling access comprises transmitting a signal to the pharmacological agent dispenser that enables the patient to dispense a pharmacological agent from the dispenser.

46. The method of claim 45 wherein the signal to the pharmacological agent dispenser unlocks the pharmacological agent dispenser.

47. The method of claim 45 wherein the signal unlocks the pharmacological agent dispenser for a finite period of time.

48. The method of claim 45 further comprising transmitting a second signal to the pharmacological agent that locks the pharmacological agent dispenser.

49. The method of claim 43 further comprising providing a communication output to a patient.

50. The method of claim 43 further comprising allowing the patient to input a communication with at least one of the device implanted in the patient's body and the device external to the patient's body.

51. The method of claim 50 wherein the communication may be used by the classifier for subsequent characterizations of the patient's propensity for future seizures.
